(19) United States
(12) Patent Application Publication
(10) Pub. No.: US 2010/0184198 A1
(43) Pub. Date: Jul. 22, 2010

(54) Systems and Methods of Urea Processing to Reduce Sorbent Load

(76) Inventors: Russell T. Joseph, Las Flores, CA (US); David J. Mishelevich, Playa del Rey, CA (US); Lina Gabrielaityte, Siauliai (LT)

Correspondence Address: PATENTMETRIX, 14252 CULVER DR. BOX 914, IRVINE, CA, 92604, US

(21) Appl. No.: 12/355,102

(22) Filed: Jan. 16, 2009

(51) Int. Cl. B01D 59/12; B01D 59/40; A62D 3/02; C25B 9/00; C12M 1/00

(52) U.S. Cl. 435/266; 95/46; 205/763; 96/6; 204/242; 435/297.1; 435/262.5

(57) ABSTRACT

The present invention provides novel methods for removal and disposal of ammonia from spent dialysate in a dialysis system. Ammonium ions present in spent dialysate are converted into gaseous ammonia by raising the pH of the spent dialysate solution in a first reactor. Gaseous ammonia diffuses through a semi-permeable hydrophobic membrane at the outlet of the first reactor and into a second reactor via a gas channel. The second reactor converts gaseous ammonia into an ammonium compound for easy disposal.

### CROSS REFERENCE

The present invention relies on U.S. Patent Provisional Application No. 60/021,987, filed on Jan. 18, 2008, for priority and is herein incorporated by reference.

### FIELD OF THE INVENTION

The present invention generally relates to the field of dialysis, and more specifically to systems and methods of urea processing in spent dialysate to effectively reduce the amount of sorbent used in cleansing the dialysate or to eliminate the need for using sorbent entirely.

### BACKGROUND OF THE INVENTION

A dialysis system typically includes a system for circulating blood, a system for circulating dialysate fluid, and a semi-permeable membrane. Urea and other blood components, but not blood cells, travel across the membrane from the blood side to the dialysate side as the blood and dialysate fluid both flow past the membrane. As dialysate fluid is recycled, urea and other blood waste compounds must be removed before the fluid is again passed by the membrane. Dialysate regeneration systems comprising closed loop multi-pass sorbent-based hemodialyzers typically use a plurality of sorbents in the form of cartridges to cleanse spent dialysate.

One way to accomplish the removal of urea in the spent dialysate is to expose the urea to urease enzyme, which breaks the urea molecules down into ammonium ions and carbonate. A sorbent type cartridge is provided in the dialysis system where urea is decomposed with the help of urease enzyme. The ammonium ions or ammonium (NH4+), which are toxic and should not be exposed to the membrane, can be adsorbed, for example, by zirconium phosphate (ZrP). In this case, zirconium phosphate acts as an ion exchanger and exchanges ammonium ions for sodium ions.

For the purpose of adsorption of ammonium ions generated by the reaction of urease enzyme breaking urea, a ZrP layer is provided in the sorbent cartridge. However the ZrP layer can only adsorb a specific quantity of ammonia while the urease enzyme can produce ammonia as long as urea is present in the blood stream. Therefore it is possible for a patient with a high urea load to produce more ammonia than the ZrP layer can adsorb. When this happens, toxic ammonia enters the dialysate and can get into the patient, which can be very harmful to the patient.

Ammonia exiting the sorbent cartridge, when the cartridge capacity to adsorb more ammonia is reached, is known as “Ammonia Breakthrough”. When this occurs, dialysis must be halted and the cartridge must be replaced.

Just as the efficiency of the ZrP sorbent to capture ammonium ions is affected after it begins to get saturated with ammonium ions, similarly, other sorbents also get used up in cleansing the spent dialysate, thereby requiring that the cartridges be periodically replaced. Sorbents are expensive, however, and it is desirable to reduce the amount of sorbents used without compromising the efficiency and effectiveness of the dialysate regeneration system to cleanse the spent dialysate. Moreover, sorbents remove desired ions, such as calcium, magnesium, and potassium from the dialysate solution. Accordingly, an additional requirement in sorbent-based systems is re-infusing ions into the dialysate after the sorbent step to ensure that the patient is not left with an electrolyte imbalance. It would therefore be preferable to remove urea without relying on sorbents to therefore avoid having to re-infuse desired ions.

Accordingly, there is need in the art for novel methods and systems of urea removal that improve the effectiveness and efficiency of the current adsorbent cartridges for dialysate reprocessing while reducing the amount of sorbent used in the process or entirely eliminating the need for using sorbent.

### SUMMARY OF THE INVENTION

According to a first object of the present invention, novel methods are provided for removal and disposal of ammonia from spent dialysate in a dialysis system. Accordingly in one embodiment, ammonium ions present in spent dialysate are converted into gaseous ammonia by raising the pH of the spent dialysate solution in a first reactor. Gaseous ammonia diffuses through a semi-permeable hydrophobic membrane at the outlet of the first reactor and into a second reactor via a gas channel. Ammonia is then captured and removed in the second reactor.

In one embodiment, ammonia is disposed of in the second reactor by electrolyzing the ammonia gas in the presence of H₂O and KOH to convert ammonia into nitrogen and hydrogen. Optionally, the hydrogen produced in this reaction is channeled to a hydrogen fuel cell. In another embodiment, ammonia is disposed off in the second reactor by first converting gaseous ammonia into an ammonium compound by mixing it with an acidic stream and then using industrial zeolite to capture the ammonium. In yet another embodiment, ammonia is removed by first converting gaseous ammonia into an ammonium compound by mixing with an acidic stream and then converting said ammonium compound into struvite mineral deposit by allowing it to react with magnesium salts and phosphorous.

In yet another embodiment, the second reactor comprises a bio-reactor, and ammonia is removed by using a microorganism for oxidation of ammonia to nitrite. In one embodiment, the microorganism is nitrosomonas europea. In still another embodiment, the second reactor comprises a three-sided horseshoe housing filled with an aqueous fluid devoid of ammonium ions. Ammonia is removed by first converting gaseous ammonia into an ammonium compound by mixing with an acidic stream and then extracting ammonium into the aqueous fluid by diffusion.

In one embodiment, the present invention comprises a method of removing ammonia from a stream of used dialysate solution in a dialysis system, the method comprising a) passing the stream of used dialysate solution having a pH through a first reactor, b) raising the pH of the stream of used dialysate solution in said first reactor to a level sufficient to substantially convert ammonium ions in said stream to gaseous ammonia, c) releasing the gaseous ammonia from said stream by allowing it to diffuse through a semi-permeable hydrophobic membrane at the outlet of said first reactor, d) receiving the gaseous ammonia through a gas channel into a second reactor, and e) capturing and removing the gaseous ammonia in said second reactor.

Optionally, the step of capturing and removing the gaseous ammonia in said second reactor further comprises converting the ammonia gas into nitrogen and hydrogen by electrolysis in the presence of H₂O and KOH. The hydrogen released in ammonia electrolysis is channeled to a hydrogen fuel cell. The step of capturing and removing the gaseous ammonia in said second reactor further comprises the steps of converting gaseous ammonia into an ammonium compound by mixing it with an acidic stream and exposing it to industrial zeolite. The step of capturing and removing the gaseous ammonia in said second reactor further comprises the steps of converting gaseous ammonia into an ammonium compound by mixing with an acidic stream and converting said ammonium compound into struvite by reacting it with magnesium salts and phosphorous.

Optionally, the second reactor is a bio-reactor and the step of capturing and removing the gaseous ammonia comprises using a microorganism, such as nitrosomonas europea, for oxidizing ammonia to nitrite. Optionally, the second reactor comprises a three-sided, e.g. horseshoe, housing and the step of capturing and removing the gaseous ammonia further comprises the steps of converting gaseous ammonia into an ammonium compound by mixing it with an acidic stream, filling said horseshoe housing with an aqueous fluid devoid of ammonium ions, and extracting ammonium into said aqueous fluid by diffusion. Optionally, the release of gaseous ammonia from the dialysate stream is assisted by a vacuum or suction device in the gas channel. Optionally, the first reactor and said second reactor are disposable.

In another embodiment, the present invention is directed to a system for removing ammonia from a stream of used dialysate solution during dialysis, the system comprising a) a first reactor through which the stream of used dialysate solution is passed and its pH raised such that ammonium ions in said stream are substantially converted to gaseous ammonia, wherein said gaseous ammonia is released from said stream by diffusion through a semi-permeable hydrophobic membrane at the outlet of said first reactor, and b) a second reactor for receiving the gaseous ammonia from the first reactor via a gas channel, wherein said second reactor captures and removes the gaseous ammonia.

Optionally, the capturing and removing the gaseous ammonia in said second reactor comprises converting the ammonia gas into nitrogen and hydrogen by electrolysis in the presence of H₂O and KOH. The hydrogen released in ammonia electrolysis is channeled to a hydrogen fuel cell. The capturing and removing the gaseous ammonia in said second reactor further comprises converting gaseous ammonia into an ammonium compound by mixing it with an acidic stream and using industrial zeolite to capture the ammonium. The capturing and removing the gaseous ammonia in said second reactor further comprises converting gaseous ammonia into an ammonium compound by mixing with an acidic stream and converting said ammonium compound into struvite by reacting with magnesium salts and phosphorous. The second reactor is a bio-reactor and capturing and removing the gaseous ammonia comprises using a microorganism for oxidizing ammonia to nitrite. The second reactor comprises a three-sided horseshoe housing and capturing and removing the gaseous ammonia further comprises converting gaseous ammonia into an ammonium compound by mixing it with an acidic stream, filling said horseshoe housing with an aqueous fluid devoid of ammonium ions, and extracting ammonium into said aqueous fluid by diffusion. The system further comprises a vacuum or suction device in the gas channel for assisting the release of gaseous ammonia from the dialysate stream.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 is a block diagram illustrating an embodiment of the ammonia release and capture system of the present invention;

FIG. 2a is a block diagram illustrating an embodiment of the first ammonia-release reactor of the ammonia release and capture system of the present invention;

FIG. 2b is a graph illustrating the ammonia stripping rate as a function of pH;

FIG. 2c is a table illustrating the ammonia stripping rate as a function of pH;

FIG. 3 is a block diagram illustrating a first embodiment of the second ammonia-capture reactor of the ammonia release and capture system of the present invention;

FIG. 4 is a block diagram illustrating a second embodiment of the second ammonia-capture reactor of the ammonia release and capture system of the present invention;

FIG. 5 is a block diagram illustrating a third embodiment of the second ammonia-capture reactor of the ammonia release and capture system of the present invention;

FIG. 6 is a block diagram illustrating a fourth embodiment of the second ammonia-capture reactor of the ammonia release and capture system of the present invention; and

FIG. 7 is a block diagram illustrating a fifth embodiment of the second ammonia-capture reactor of the ammonia release and capture system of the present invention.

### DETAILED DESCRIPTION OF THE INVENTION

While the present invention may be embodied in many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein, which would be evident to one of ordinary skill in the art are contemplated and incorporated herein.

The present invention is directed towards novel methods and systems for removing ammonia in closed loop multi-pass sorbent based hemodialysis systems, such as portable or wearable kidney dialysis devices. The system strips off gaseous ammonia from the dialysate and then disposes of the gaseous ammonia using novel methods.

Dialysate is regenerated for reuse in multi-pass dialysis systems by passing it through a regeneration section comprising a plurality of sorbent cartridges and suitable additives. A typical sorbent cartridge system comprises a urease cartridge, a zirconium phosphate cartridge, a hydrous zirconium oxide cartridge and an activated carbon cartridge. Those of ordinary skill in the art will recognize that these sorbents are similar to the sorbents employed by the commercially available REDY™ System.

The principle of the sorbent cartridge system is based on hydrolysis of urea to ammonium carbonate by the enzymatic reaction of urease. The ammonia and ammonium ions are then removed by the zirconium phosphate (NaHZrP) in exchange for hydrogen ions and Na⁺ ions. The enzymatic conversion of urea in the urease cartridge causes one mole of urea to be decomposed into two moles of ammonia and one mole of carbon dioxide by way of the following reaction:

CO(NH₂)₂+3H₂O [symbol] 2NH₄⁺+CO₂+2OH⁻

Ammonia (NH₃) is primarily (>95%) present as ammonium ion (2NH₄⁺), since its pKa of 9.3 is substantially greater than the solution pH.

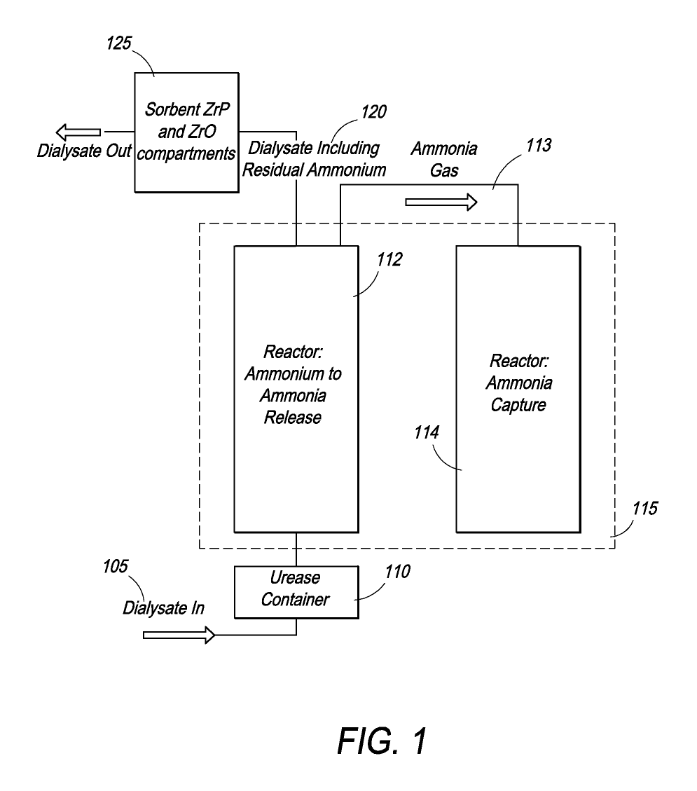

FIG. 1 shows a block diagram illustration of the present invention where spent dialysate, comprising uremic wastes including urea, is pumped through a plurality of sorbent cartridges for cleansing. As spent dialysate 105 passes through the urease cartridge 110, an enzymatic reaction resulting in hydrolysis of urea causes release of ammonia and ammonium ions apart from other by-products as discussed earlier. According to a novel aspect of the present invention the dialysate emanating from the urease cartridge 110 and comprising ammonia, substantially in the form of ammonium ions, is passed through an ammonia release and capture stage 115. As shown in FIG. 1, the ammonia release and capture stage 115 of the present invention, in one embodiment, comprises a first ammonia-release reactor 112 where the ammonium ions in the dialysate are released as ammonia gas, which in turn is made to pass through an ammonia gas channel 113 to a second ammonia-capture reactor 114 where the ammonia gas is captured. The dialysate 120 emanating from the ammonia release and capture stage 115 is substantially stripped of ammonia/ammonium ions. The dialysate 120 with any residual ammonium ions flows onwards through subsequent sorbent cartridges 125 comprising layers of adsorbent materials such as ZrP and ZrO for further cleansing.

According to an aspect of the present invention the first and second reactors, 112, 114, of the ammonia release and capture stage 115 are air-tight canisters that in one embodiment are disposable. In one embodiment, the first ammonia-release reactor 112 is a daily disposable canister while the second ammonia-capture reactor 114 is a weekly or monthly disposable canister or an even further long-term durable canister. In one embodiment, the first ammonia-release reactor 112 is a daily, weekly, monthly, or an even longer term disposable canister while the second ammonia-capture reactor 114 is a daily, weekly, monthly or an even longer-term disposable canister.

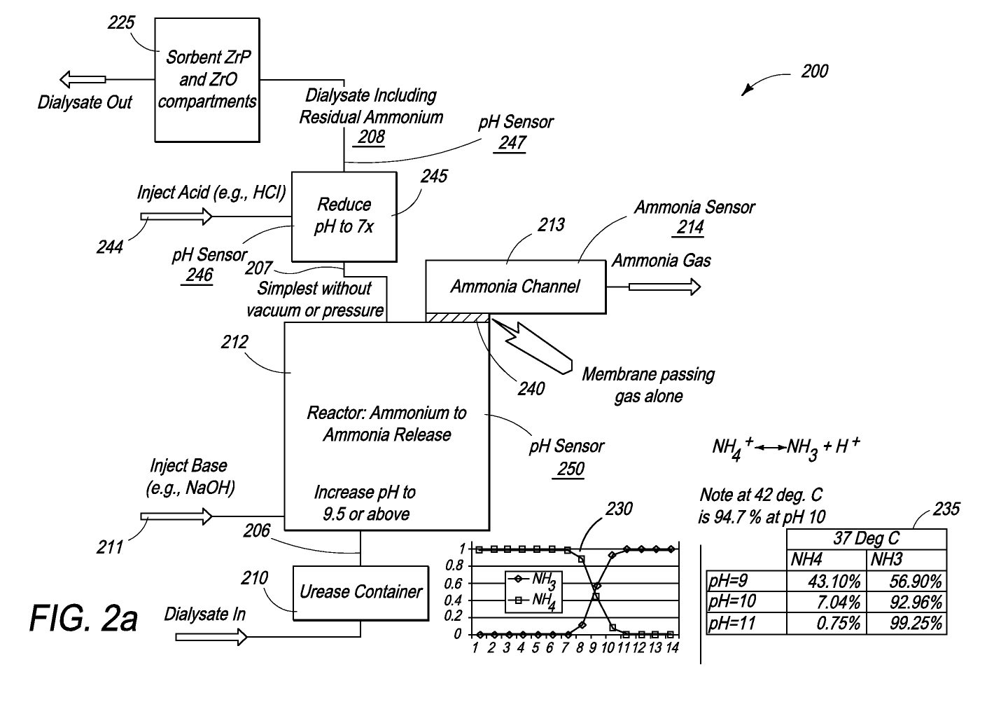

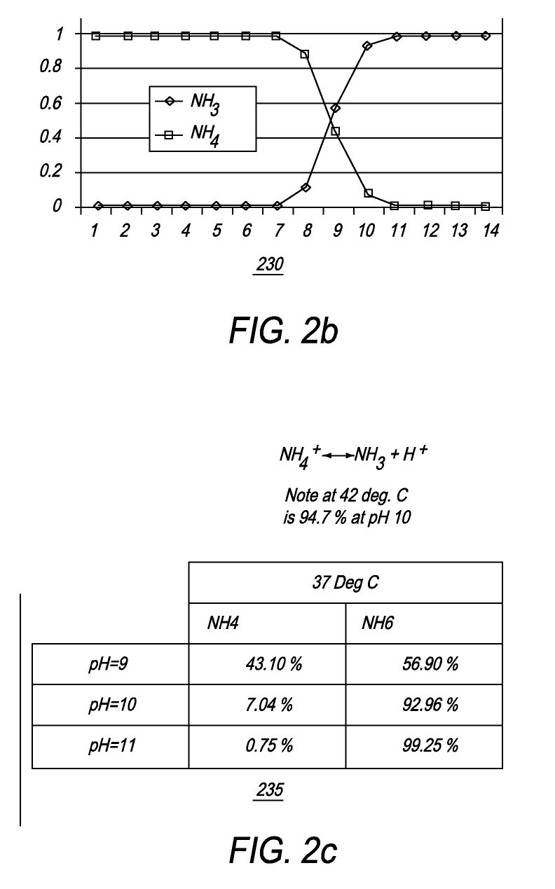

FIG. 2a shows a block diagram illustrating an embodiment 200 of the first ammonia-release reactor 112 of the ammonia release and capture stage 115 of FIG. 1. Turning to FIG. 2a, the stream of dialysate 206 emanating from the urease cartridge 210 and comprising ammonium ions is processed in the first ammonia-release reactor 212 for the ammonium ion conversion. The ammonium ion conversion comprises sufficiently increasing the pH of the dialysate stream 206 to convert ammonium in the stream to gaseous ammonia. The dialysate stream 206 in the first reactor 212 is contacted with a weak and/or strong base 211, such as sodium hydroxide (NaOH) or potassium hydroxide (KOH) to raise the pH level above 9.5. That is, it is ensured that there is sufficient alkalinity available to supply the necessary equivalents to maintain a pH above 9.5 as the ammonia dissociates (NH₄+ [symbol] NH₃+H⁺) and is stripped. A pH sensor 250 is connected to the first reactor 212 to monitor and maintain its pH level.

At pH levels above 9.5, the ammonia fraction is largely gaseous ammonia and is readily stripped from the dialysate stream 206. The ammonia stripping rate is a function of the pH level as well as the temperature apart from other parameters such as the available surface area for the reaction in the reactor 212. In one embodiment the temperature of the dialysate stream in the first reactor 212 is about 37 degrees C. The reaction is further depicted in graph 230 and table 235, which are described in detail later in the specification, with reference to FIGS. 2b and 2c.

Referring back to FIG. 2a, the first reactor 212 has a vent that is sealed with a microporous semi-permeable hydrophobic membrane 240 that allows gaseous ammonia to diffuse through but does not allow the aqueous dialysate stream 206 to pass. Ammonia gas diffusing through the hydrophobic membrane 240 is collected via an ammonia channel 213 for further processing. An ammonia sensor 214 is advantageously connected to the ammonia channel/collection device 213. In one embodiment the ammonia diffusing through the membrane 240 is collected without any vacuum or pressure. In another embodiment a venturi is used, at the ammonia channel 213, such that ammonia gas is withdrawn from the first reactor 212 under vacuum into the suction side of the venturi for onward communication and further processing. The venturi associated with the first reactor 212 provides a nearly full vacuum on the first reactor 212 thus allowing for rapid and nearly complete separation of ammonia gas from the dialysate stream 206. In an alternate embodiment an impeller (not shown) is used in the ammonia channel 213 to suction out ammonia gas from the first reactor 212 through the hydrophobic membrane 240.

The aqueous dialysate stream 207 comprising residual ammonia flows through an opening into an auxiliary air-tight canister 245. An acid 244, such as hydrochloric acid (HCl) is injected into the auxiliary canister 245 lowering the pH level of the dialysate stream 207 to about 7. At such lowered pH levels the residual ammonia is converted to ammonium ions that remain in aqueous state dissolved in the dialyate stream 207. First and second pH sensors, 246, 247, are advantageously connected to monitor the pH level of the dialysate stream 207 in the canister 245 and the stream 208 flowing out therefrom. The dialysate 208 with residual ammonium ions flows onwards through subsequent sorbent cartridges 225 such as ZrP, ZrO for further cleansing.

Referring to FIG. 2b, graph 230 depicts that beyond a pH level of 8.5 the equilibrium rapidly shifts in the direction of gaseous ammonia (NH₃). The graph 230 corresponds to the readings in table 235 shown in FIG. 2c. Referring to FIG. 2c, table 235 provides ammonium and ammonia levels at pH levels of 9, 10 and 11 at a dialysate stream temperature of 37 degrees C. It is apparent from table 235 and graph 230, that at pH of 10 and temperature of 37 degrees C. the level of ammonia gas is 92.96%.

It should be appreciated that the system can regenerate dialysate without requiring the use of a final sorbent stage. Therefore, in another embodiment, the system of the present invention does not employ a residual sorbent stage, thereby eliminating sorbent 125 (FIG. 1) and 225 (FIG. 2). By eliminating the sorbent, the need for re-infusing magnesium, calcium, and potassium, or other desirable ions, minerals, or nutrients, into the dialysate solution is not required. It should be appreciated that all of the embodiments disclosed herein further include a system wherein the use of sorbents, or inclusion of a sorbent-based regeneration phase, is eliminated.

Additionally, it should be appreciated that each of the disclosed embodiments further include a version wherein the conversion of ammonium to ammonia is facilitated by increasing temperature of the dialysate fluid above 37 degrees Celsius (e.g., at 42 degrees Celsius there is 94.7% ammonia conversion; at 37 degrees Celsius there is 92.96% ammonia conversion), and then cooling the dialysate down again to 37 degrees Celsius prior to the dialysate fluid passing through the dialyzer.

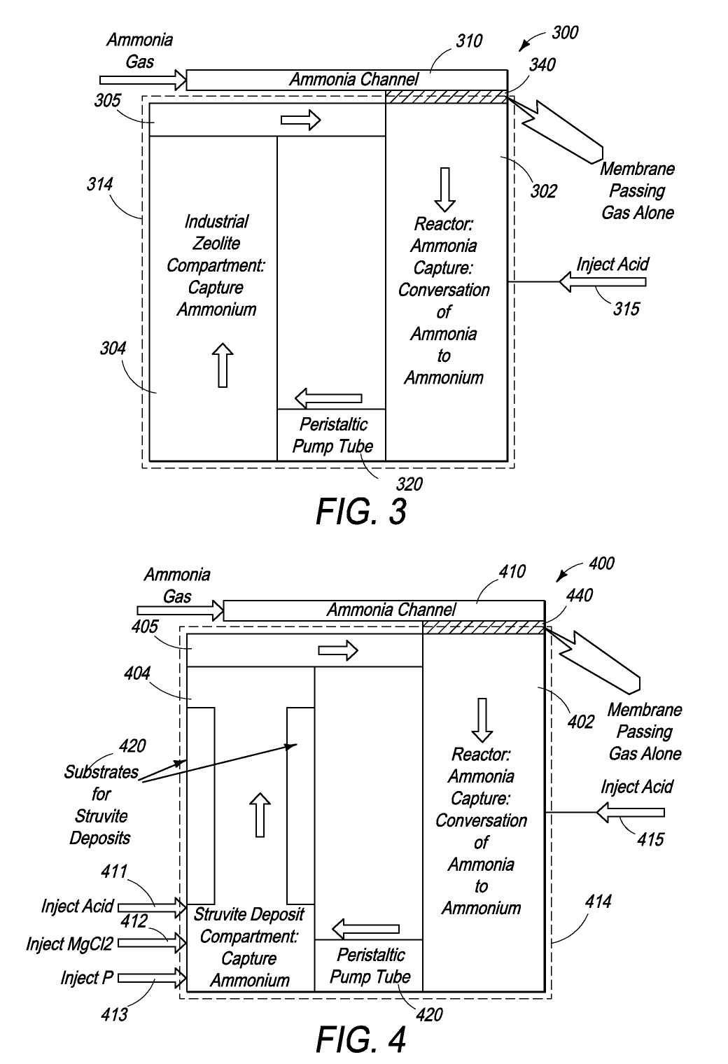

FIG. 3 shows a block diagram illustrating a first embodiment 300 of the second ammonia-capture reactor 114 of the ammonia release and capture stage 115 of FIG. 1. The ammonia gas diffusing out from the first ammonia-release reactor or suctioned by a venturi, as described with reference to FIG. 2a, and is then passed through the second ammonia-capture reactor 314 for ammonia removal/capture via ammonia channel 310. The ammonia gas is received in a first compartment 302 of the reactor 314 through an inlet that is sealed with a hydrophobic membrane 340. The hydrophobic membrane 340 allows gases to diffuse through but prevents aqueous fluids from passing. Ammonia gas is converted by mixing the gas with an acidic stream 315, such as sulfuric acid, that is injected into the compartment 302. In one embodiment, the pH of the contents of the first compartment 302 is maintained in the range from 6 to 7. At such reduced pH levels, the ammonia gas reacts with the acid 315 to form an ammonium compound. As the acidic stream 315 becomes saturated with NH₄, a solution of the ammonium compound is obtained. For example, if the acidic stream is that of sulfuric acid, then an ammonium sulfate solution is obtained by the reaction of ammonia gas with the acid. The obtained solution is pumped using a peristaltic pump, through a tube, 320, into a second compartment 304, which comprises a pack or column of an industrial zeolite compound. As the solution percolates through the zeolite pack/column the ammonium ions are captured by the zeolite while the resultant solution, substantially stripped of ammonium ions and comprising any residual ammonium, is circulated back to the first compartment 302 through tube 305. In one embodiment the zeolite pack/column is comprised in an auxiliary canister/cartridge that is removably attached to the second compartment 304 as a screw-on container.

FIG. 4 shows a block diagram illustrating a second embodiment 400 of the second ammonia-capture reactor 114 of the ammonia release and capture stage 115 of FIG. 1. The ammonia gas diffusing out of, or suctioned from, the first ammonia-release reactor, as described with reference to FIG. 2a, is passed through the second ammonia-capture reactor 414 for ammonia removal/capture via ammonia channel 410. The ammonia gas is received in a first compartment 402 of the reactor 414 through an inlet that is sealed with a hydrophobic membrane 440 (that allows gases to diffuse through but prevents aqueous fluids from passing) for conversion to NH₄. Ammonia gas is converted by mixing the gas with an acidic stream 415, such as sulfuric acid, that is injected into the compartment 402 through an inlet.

In one embodiment the pH of the contents of the first compartment 402 is maintained in the range from 6 to 7. At such reduced pH levels, the ammonia gas reacts with the acid 415 to form an ammonium compound. As the acidic stream 415 becomes saturated with the NH₄, the ammonium compound solution (such as ammonium sulfate solution in case the acidic stream is that of sulfuric acid) is pumped by a peristaltic pump, through a tube 420, into a compartment 404, where it is converted to an insoluble mineral deposit such as struvite. As the solution is pumped through compartment 404 streams of acid 411 (such as sulfuric acid), Mg⁺⁺ ions 412 (in the form of magnesium salts such as MgCl₂, MgO) and phosphorus 413 are injected into the compartment 404 for mixing with the pumped solution. Ammonium and magnesium combine with phosphorous in a 1:1:1 molar ratio to form an insoluble mineral struvite as follows:

NH₄⁺+Mg²⁺+PO₄³⁻+6H₂O→NH₄MgPO₄.6H₂O (struvite)

The struvite gets deposited on substrates 420 in the compartment 404 in the form of large crystals and may be removed periodically. As the solution percolates through the compartment 404 the ammonium ions are captured and precipitated out in the form of struvite, while the resultant solution, substantially stripped of ammonium ions and comprising any residual ammonium, is circulated back to the first compartment through tube 405.

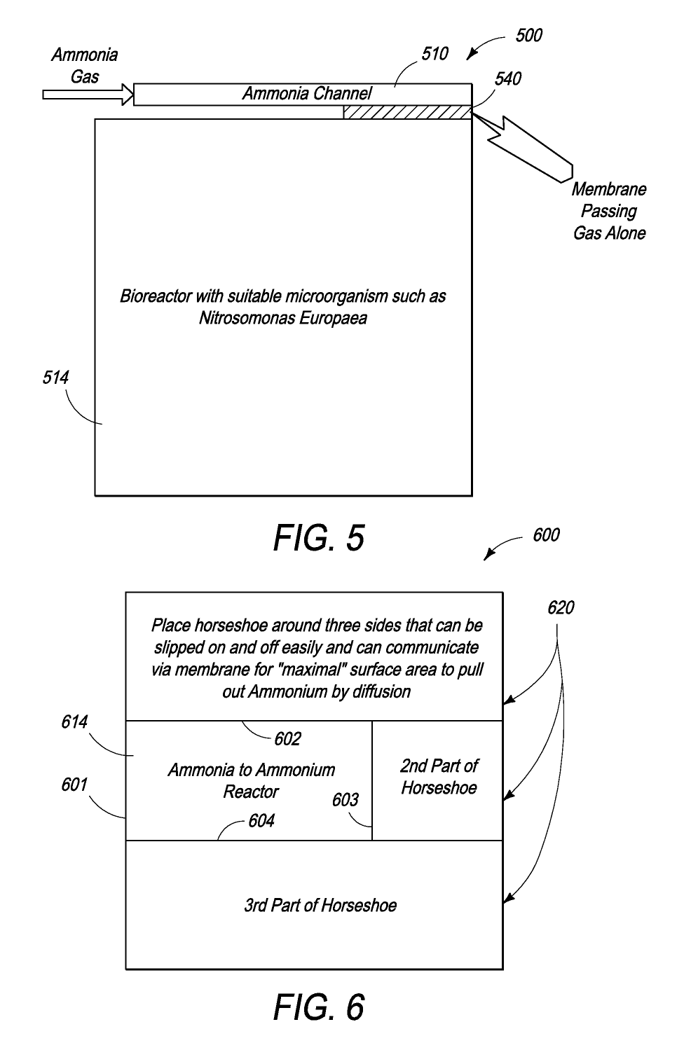

FIG. 5 shows a block diagram illustrating a third embodiment 500 of the second ammonia-capture reactor 114 of the ammonia release and capture stage 115 of FIG. 1. The ammonia gas diffusing out from the first ammonia-release reactor or suctioned by a venturi, as described with reference to FIG. 2a, is passed through the second ammonia-capture reactor 514 for ammonia removal/capture via ammonia channel 510. The ammonia gas is received in the reactor 514 through an inlet that is sealed with a hydrophobic membrane 540 (that allows gases to diffuse through but prevents aqueous fluids from passing). The reactor 514 is a bio-reactor comprising suitable micro-organisms that feed on ammonia to organically capture and convert ammonia. In one embodiment the micro-organism is nitrosomonas europeae. As would be known to persons of ordinary skill in the art nitrosomonas europea is a Gram-negative obligate chemolithoautotroph that can derive all its energy and reductant for growth from the oxidation of ammonia to nitrite. This microbe prefers an optimum pH of 6.0 to 9.0, fairly neutral conditions, has an aerobic metabolism and prefers a temperature range of 20 to 30 degrees Celsius.

FIG. 6 shows a block diagram illustrating a fourth embodiment 600 of the second ammonia-capture reactor 114 of the ammonia release and capture stage 115 of FIG. 1. Ammonia gas diffusing out from the first ammonia-release reactor or suctioned by a venturi, as described with reference to FIG. 2a, is passed through the second reactor 614 for ammonia capture. The ammonia gas is received in the reactor 614 through an inlet, at a first side 601 that is sealed with a hydrophobic membrane, that allows gases to diffuse through but prevents aqueous fluids to pass, for conversion to NH₄. Ammonia gas is converted by mixing the gas with an acidic stream, such as sulfuric acid, that is injected into the reactor 614. In one embodiment, the pH of the contents of the reactor 614 is maintained in the range from 6 to 7. At such reduced pH levels, the ammonia gas reacts with the acid to form an ammonium compound. In one embodiment the other three sides, 602, 603 and 604, of the reactor 614 are partially or completely made of a semi-permeable membrane that allows solutes and other compounds in aqueous solutions to diffuse through due to osmotic pressure differentials.

A module 620 conformed as a horseshoe, or U-shaped, housing is capable of being removably slipped onto the reactor 614 such that the horseshoe housing covers the three sides, 602, 603 and 604, of the reactor 614 comprising the semi-permeable membranes. The housing, in one embodiment, comprises an inlet from where an aqueous fluid, such as water, devoid of ammonium ions is introduced in the horseshoe housing to completely fill it. The aqueous fluid in the horseshoe housing communicates with the ammonium solution within the reactor to extract ammonium by diffusion.

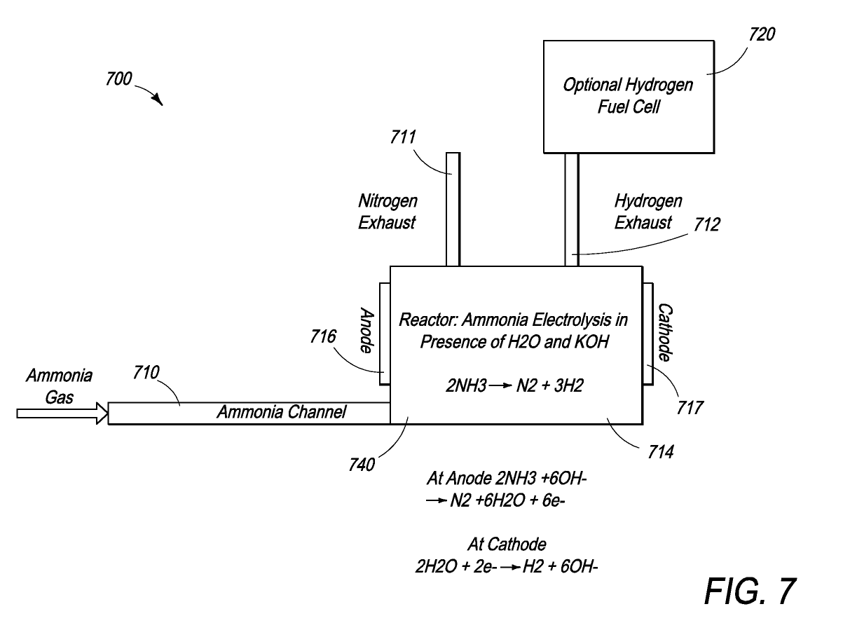

FIG. 7 shows a block diagram illustrating a fifth embodiment 700 of the second ammonia-capture reactor 114 of the ammonia release and capture stage 115 of FIG. 1. Ammonia gas diffusing out from the first ammonia-release reactor or suctioned by a venturi, as described with reference to FIG. 2a, is passed through the second ammonia-capture reactor 714, via ammonia channel 710, for electrolysis. The ammonia gas is received in the reactor 714 through an inlet 710, which is sealed with a hydrophobic membrane 740 (that allows gases to diffuse through but prevents aqueous fluids from passing). The reactor 714 comprises an anode 716 and cathode 717 at two opposing sides. First and second exhausts 711, 712 are provided on a second side of reactor 714 such that they are proximal to the anode 716 and cathode 717 respectively. The reactor 714 comprises an aqueous base, such as potassium hydroxide (KOH), as an electrolyte such that electrolysis of ammonia occurs in the presence of H₂O and KOH as follows:

At anode: 2NH₃+6OH⁻→N₂+6H₂O+6e⁻

At cathode: 2H₂O+2e⁻→H₂+6OH⁻

The resulting N₂ at the anode is vented out through first exhaust 711 while the H₂ is let out via second exhaust 712. In one embodiment the second exhaust 712 venting H₂ is optionally connected to a Hydrogen Fuel Cell 720 that uses the vented hydrogen as fuel.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the appended claims.

We claim:

1. A method of removing ammonia from a stream of used dialysate solution in a dialysis system, the method comprising:
passing the stream of used dialysate solution having a pH through a first reactor;
raising the pH of the stream of used dialysate solution in said first reactor to a level sufficient to substantially convert ammonium ions in said stream to gaseous ammonia;
releasing the gaseous ammonia from said stream by allowing it to diffuse through a semi-permeable hydrophobic membrane at the outlet of said first reactor;
receiving the gaseous ammonia through a gas channel into a second reactor; and
capturing and removing the gaseous ammonia in said second reactor.

2. The method of claim 1, wherein the step of capturing and removing the gaseous ammonia in said second reactor further comprises converting the ammonia gas into nitrogen and hydrogen by electrolysis in the presence of H₂O and KOH.

3. The method of claim 2, wherein the hydrogen released in ammonia electrolysis is channeled to a hydrogen fuel cell.

4. The method of claim 1, wherein the step of capturing and removing the gaseous ammonia in said second reactor further comprises the steps of converting gaseous ammonia into an ammonium compound by mixing it with an acidic stream and exposing it to industrial zeolite.

5. The method of claim 1, wherein the step of capturing and removing the gaseous ammonia in said second reactor further comprises the steps of converting gaseous ammonia into an ammonium compound by mixing with an acidic stream and converting said ammonium compound into struvite by reacting it with magnesium salts and phosphorous.

6. The method of claim 1, wherein said second reactor is a bio-reactor and the step of capturing and removing the gaseous ammonia comprises using a microorganism for oxidizing ammonia to nitrite.

7. The method of claim 6, wherein said microorganism is nitrosomonas europea.

8. The method of claim 1, wherein said second reactor comprises a three-sided horseshoe housing and the step of capturing and removing the gaseous ammonia further comprises the steps of converting gaseous ammonia into an ammonium compound by mixing it with an acidic stream, filling said horseshoe housing with an aqueous fluid devoid of ammonium ions, and extracting ammonium into said aqueous fluid by diffusion.

9. The method of claim 1, wherein the release of gaseous ammonia from the dialysate stream is assisted by a vacuum or suction device in the gas channel.

10. The method of claim 1, wherein said first reactor and said second reactor are disposable.

11. A system for removing ammonia from a stream of used dialysate solution during dialysis, the system comprising:
a first reactor through which the stream of used dialysate solution is passed and its pH raised such that ammonium ions in said stream are substantially converted to gaseous ammonia, wherein said gaseous ammonia is released from said stream by diffusion through a semi-permeable hydrophobic membrane at the outlet of said first reactor; and
a second reactor for receiving the gaseous ammonia from the first reactor via a gas channel, wherein said second reactor captures and removes the gaseous ammonia.

12. The system of claim 11, wherein capturing and removing the gaseous ammonia in said second reactor comprises converting the ammonia gas into nitrogen and hydrogen by electrolysis in the presence of H₂O and KOH.

13. The system of claim 12, wherein the hydrogen released in ammonia electrolysis is channeled to a hydrogen fuel cell.

14. The system of claim 11, wherein capturing and removing the gaseous ammonia in said second reactor further comprises converting gaseous ammonia into an ammonium compound by mixing it with an acidic stream and using industrial zeolite to capture the ammonium.

15. The system of claim 11, wherein capturing and removing the gaseous ammonia in said second reactor further comprises converting gaseous ammonia into an ammonium compound by mixing with an acidic stream and converting said ammonium compound into struvite by reacting with magnesium salts and phosphorous.

16. The system of claim 11, wherein said second reactor is a bio-reactor and capturing and removing the gaseous ammonia comprises using a microorganism for oxidizing ammonia to nitrite.

17. The system of claim 16, wherein said microorganism is nitrosomonas europea.

18. The system of claim 11, wherein said second reactor comprises a three-sided horseshoe housing and capturing and removing the gaseous ammonia further comprises converting gaseous ammonia into an ammonium compound by mixing it with an acidic stream, filling said horseshoe housing with an aqueous fluid devoid of ammonium ions, and extracting ammonium into said aqueous fluid by diffusion.

19. The system of claim 11 further comprising a vacuum or suction device in the gas channel for assisting the release of gaseous ammonia from the dialysate stream.

20. The system of claim 11, wherein said first reactor and said second reactor are disposable.
